(19) United States
(12) Patent Application Publication
(10) Pub. No.: US 2021/0128894 A1
(43) Pub. Date: May 6, 2021

(54) System and Method for Accelerating Cervical Ripening

(71) Applicant: Alpha Phase Technologies LLC, Lancaster, OH (US)

(72) Inventors: David D. Burnette, Lancaster, OH (US); Ariel L. Lanier, Lancaster, OH (US)

(73) Assignee: Alpha Phase Technologies LLC, Lancaster, OH (US)

(21) Appl. No.: 16/578,168

(22) Filed: Sep. 20, 2019

(51) Int. Cl. A61M 29/02

(52) U.S. Cl. CPC A61M 29/02; A61M 2205/10; A61M 2205/3331; A61M 2210/14

(57) ABSTRACT

A cervical ripening system includes a balloon catheter and a fluid pump. The fluid catheter has a balloon portion configured to pass through a cervix when contracted and to engage and pressure a uterine side of the cervix when expanded by fluid. The fluid pump is in fluid flow communication with the balloon portion of the catheter and is configured to selectively expand and contract the balloon portion of the balloon catheter by alternately pumping fluid into and out of the balloon portion of the balloon catheter to repetitively increase and decrease pressure on the uterine side of the cervix to simulate natural uterine contractions.

### CROSS-REFERENCE TO RELATED APPLICATIONS

Not Applicable

### STATEMENT REGARDING FEDERALLY SPONSORED RESEARCH

Not Applicable

### PARTIES TO A JOINT RESEARCH AGREEMENT

Not Applicable

### REFERENCE TO APPENDIX

Not Applicable

### FIELD OF INVENTION

The field of invention generally relates to induction of labor, and, more particularly, to systems and methods for cervical ripening which reduce the length of time to achieving cervical favorability and thus time to delivery, while avoiding increased uterine stimulation.

### BACKGROUND OF THE INVENTION

In the United States, more than 22% of pregnant women undergo labor induction. The goal of labor induction is to achieve vaginal delivery of an infant by stimulating uterine contractions before the onset of spontaneous labor. There are various methods used to facilitate induction of labor which arc used individually, or most frequently, in combination. These methods include synthetic oxytocin administration (Pitocin), membrane “stripping” or “sweeping”, artificial rupture of membranes, cervical ripening by pharmacological methods, and cervical ripening by mechanical methods. Favorability of the cervix informs the method of induction. Successful vaginal delivery is more often achieved if the cervix is favorable prior to the administration of Pitocin. Length of time to delivery is also shorted when the favorability of the cervix is increased, with associated reductions in total Pitocin use. Prolonged use of Pitocin is associated with an increased incidence of uterine tachysystole, fetal distress, maternal hyponatremia, and postpartum hemorrhage.

Oxytocin is a peptide hormone released by the posterior pituitary that binds to uterine receptors to produce rhythmic constriction of the uterine muscles, or contractions. Oxytocin encourages cervical remodeling by a combination of direct chemical effects on the cervix and by causing mechanic stress to the cervix through contractions. The synthetic analogue of oxytocin is Pitocin. Pitocin is the most commonly used induction agent, the risk of which is discussed above.

Membrane stripping or sweeping involves inserting the examiner's finger beyond the internal cervical os and then rotating the finger circumferentially along the lower uterine segment to detach the fetal membranes from the decidua. This mechanical disruption is thought to cause the release of certain chemicals, hormones, and vasoactive peptides responsible for cervical remodeling. The patient must already have a cervical dilation of 1-2 cm in order to allow passage of a digit. The efficacy of membrane stripping has not been shown in the literature, and may cause bleeding, premature rupture of fetal membranes, and possible increase risks of infections.

Artificial rupture of fetal membranes, or amniotomy, can be used in isolation or, more commonly, in conjunction with, Pitocin administration. Similar to membrane stripping, amniotomy can only be performed if the patient has a baseline level of cervical dilation. Amniotomy is thought to aid in the induction process by exposing the cervix to the chemical milieu of the amniotic cervix to encourage remodeling, and allowing enhanced contractility of the uterine walls against the fetus to aid in descent of the fetus into the pelvis. Risk of amiotomy include prolapse of the umbilical cord, placental abruption, and increase risk of intrauterine infection with increase time from membrane rupture to delivery.

Pharmacologic cervical ripening agents arc prostaglandin analogues, of which there are two; dinoprostone (PGE2) and misoprostol (PGE1). Dinoprostone exists in gel form (Prepidil), suppository form (Prostin E2) and as a vaginal insert (Cervidil). All forms of dinoprostone are FDA approved for use as a cervical ripening agent for labor induction. Misoprostol (Cytotec) in tablet from, while not FDA approved for this use, is a common agent for cervical ripening.

All pharmacologic cervical ripening agents concurrently cause uterine stimulation in the form of contractions. Frequently the uterus becomes hyper-stimulated, with uterine contraction frequency of greater than 5 contractions in 10 minutes, or tachysystole. This may result in fetal distress. Continuous fetal and uterine activity monitoring is required during use of these agents due to risk of tachysystole and associated fetal distress. While the Cervidil insert is easily removed if either of these conditions arises, the prostaglandin gel, suppository, and tablet cannot be reversed.

There are two main types of mechanical cervical ripeners; osmotic dilators and balloon catheters. Osmotic dilators arc less frequently used in the United States than either pharmacologic methods or balloon catheters. Two osmotic dilators are currently on market. Dilateria is an organic product made from a specific species of dried and sterilized seaweed, Laminaria Japonica. Dilapan-S is a synthetic analogue, made of hydrophilic polymers. Both devices after being inserted into the cervical canal by a physician work by absorbing fluid and expanding, exerting internal lateral force on the cervix, causing primarily dilation. A significant increase in infection rates have been reported with the organic product, as well as allergic reactions. Dilation across the device is also variable, and adequate cervical dilation may not be achieved. These products are also brittle, and breakage of the devices during placement or extraction is possible.

The most commonly used balloon catheters are Foley catheters and Cook catheters. The balloon of a Foley catheter is moved through the opening in the cervix so that the balloon is located just beyond the cervix, but outside the amniotic sac. The balloon is then inflated by inserting sterile water. Often the outer end of the catheter is taped to the woman's thigh to create additional tension. With the balloon inflated, it puts pressure on the internal cervical os, helping the cervix dilate and increasing the tissue's response to oxytocin and prostaglandins. The balloon catheter usually falls out when the cervix has dilated about 3 cm. Use of Cook catheters is similar to Foley catheters but Cook catheters have two balloons. The first balloon is moved through the opening in the cervix so that the balloon is located just beyond the cervix, but outside the amniotic sac. The first balloon is then inflated by inserting sterile water. The second balloon, which is located outside the cervix, is then inflated by inserting sterile water. With both of the balloons inflated, the first balloon puts pressure on the internal cervical os and the second balloon puts pressure on the external cervical os so that no additional traction is needed.

Spontaneous labor is a complex interplay between both chemical and mechanical forces. Many combinations of induction methods have been studied. For example, concurrent use of Pitocin with a balloon catheter and the simultaneous use of prostaglandins with balloon catheters arc common practice. While these methods may decrease interval time to delivery compared to stepwise use of a mechanical ripening agent followed by a contractile agent, they increase the total time of uterine stimulation.

Accordingly, there is a need for improved systems and methods for accelerating cervical ripening.

### SUMMARY OF THE INVENTION

Disclosed arc systems and methods for accelerating cervical ripening that overcome at least some of the above-described problems associated with the prior art. Disclosed is a cervical ripening system comprising, in combination, a balloon catheter and a fluid pump. The fluid catheter has a balloon portion configured to pass through a cervix when contracted and to engage and pressure a uterine side of the cervix when expanded by a fluid. The fluid pump is in fluid flow communication with the balloon portion of the catheter and is configured to selectively expand and contract the balloon portion of the balloon catheter to repetitively increase and decrease pressure on the uterine side of the cervix.

Also disclosed is a cervical ripening system comprising, in combination, a balloon catheter and at least one fluid pump. The balloon catheter has a first balloon portion configured to pass through a cervix when contracted and to engage and pressure a uterine side of the cervix when expanded by a fluid and a second balloon portion configured to pass to the cervix when contracted and to engage a vaginal side of the cervix when expanded by a fluid. The at least one fluid pump is in fluid flow communication with the first balloon portion and the second fluid portion of the catheter and is configured to selectively expand and contract at least one of the first and second balloon portions of the balloon catheter to repetitively increase and decrease pressure on the uterine side of the cervix.

Also disclosed is a method for cervical ripening comprising the steps of, in combination, passing a balloon portion of a balloon catheter through a cervix and into a uterus when the balloon portion is contracted, and expanding and contracting the balloon portion of the balloon catheter within the uterus with a fluid using a fluid pump in fluid flow communication with the balloon portion of the balloon catheter to repetitively increase and decrease pressure on the uterine side of the cervix.

From the foregoing disclosure and the following more detailed description of various preferred embodiments it will be apparent to those skilled in the art that the present invention provides a significant advance in the technology and art of systems and methods for cervical ripening. Particularly significant in this regard is the potential the invention affords for reducing the time for cervical ripening, and thus the total time of uterine stimulation, in a simple and cost effective manner. Additional features and advantages of various preferred embodiments will be better understood in view of the detailed description provided below.

### BRIEF DESCRIPTION OF THE DRAWINGS

These and further features of the present invention will be apparent with reference to the following description and drawing, wherein:

FIG. 1 is a schematic view of a system for accelerating cervical ripening according to a first embodiment of the present invention.

FIG. 2 is a perspective view of a balloon catheter of the system of FIG. 1.

FIG. 3 is a diagrammatic view of the balloon catheter of FIG. 2 inserted through a cervix.

FIG. 4A is a diagrammatic view of a force profile for uterine contractions;

FIGS. 4B to 4E are a diagrammatic views of exemplary force profiles for the balloon catheter of the system of FIG. 1.

FIG. 5 is a schematic view of a system for accelerating cervical ripening according to a second embodiment of the present invention.

FIG. 6 is a perspective view of a balloon catheter of the system of FIG. 5.

FIG. 7 is a schematic view of a system for accelerating cervical ripening according to a third embodiment of the present invention.

It should be understood that the appended drawings are not necessarily to scale, presenting a somewhat simplified representation of various preferred features illustrative of the basic principles of the invention. The specific design features of the cervical ripening systems as disclosed herein, including, for example, specific dimensions and shapes of the various components will be determined in part by the particular intended application and use environment. Certain features of the illustrated embodiments have been enlarged or distorted relative to others to facilitate visualization and clear understanding. In particular, thin features may be thickened, for example, for clarity or illustration. All references to direction and position, unless otherwise indicated, refer to the orientation of the components illustrated in the drawings.

### DETAILED DESCRIPTION OF CERTAIN PREFERRED EMBODIMENTS

It will be apparent to those skilled in the art, that is, to those who have knowledge or experience in this area of technology, that many uses and design variations are possible for the systems and methods for accelerating cervical ripening disclosed herein. The following detailed discussion of various alternative and preferred embodiments will illustrate the general principles of the invention. Other embodiments suitable for other applications will be apparent to those skilled in the art given the benefit of this disclosure.

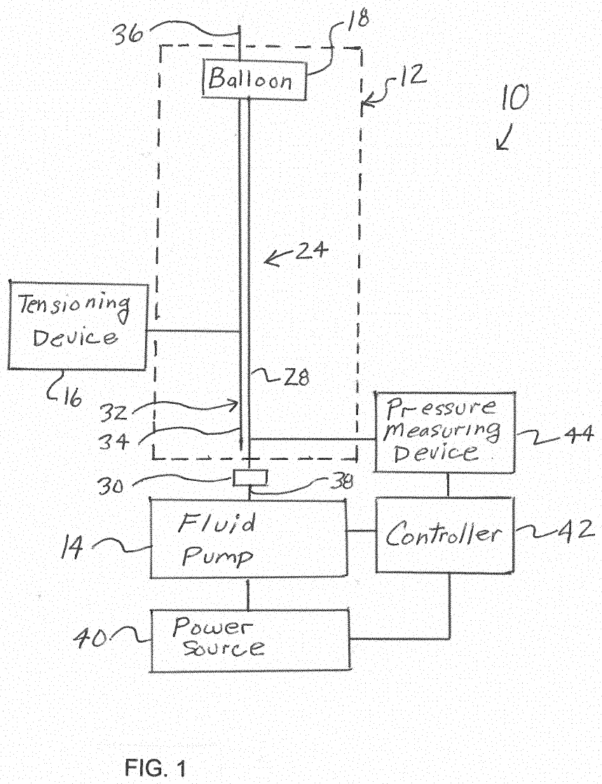

FIGS. 1 to 3 illustrate a cervical ripening system 10 for accelerating cervical ripening according to a first embodiment of the invention. The illustrated cervical ripening system 10 includes a balloon catheter 12, a motorized fluid pump 14, and a traction or tension device 16. The balloon catheter 12 has a balloon portion 18 configured to pass through a cervix 20 when contracted and to engage and pressure a uterine side 22 of the cervix 20 when expanded by a fluid. The motorized fluid pump 14 is in fluid flow communication with the balloon portion 18 of the balloon catheter 12 and is configured to selectively expand and contract the balloon portion 18 of the balloon catheter 12 to repetitively pressure the uterine side 22 of the cervix 20 to simulate forces which occur during natural uterine contractions.

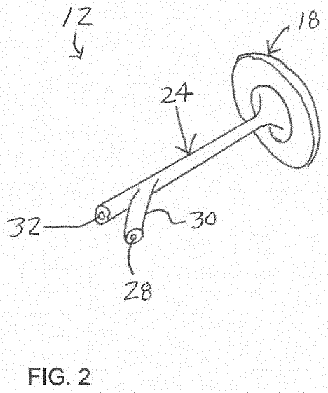

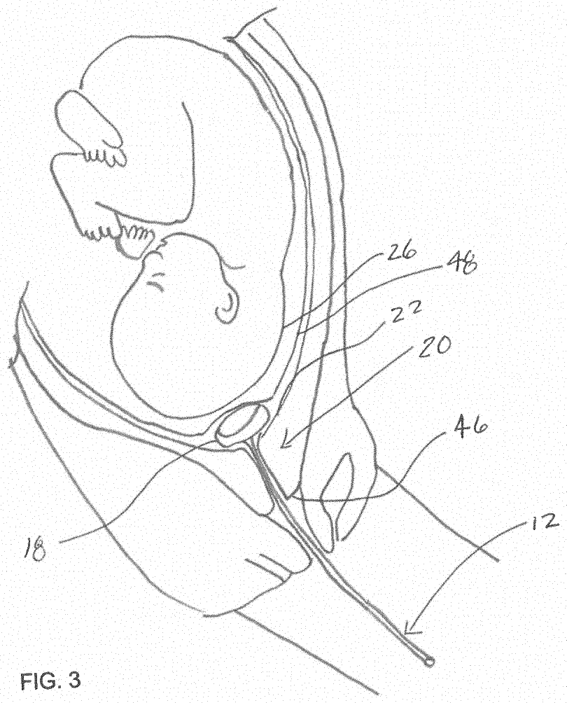

An exemplary balloon catheter 12 having the balloon portion 18 and a tube portion 24 (best seen in FIG. 2). The illustrated balloon portion 18, when inflated, is relatively flat in order to more fully engage the uterine side 22 of the cervix 20 and to minimize interference with the baby's head 26 (best seen in FIG. 3). However, the balloon portion 18 can alternatively have any other suitable shape such as, for example but not limited to, spherical, semi-spherical, cylindrical, oblong or egg-shaped, heart or turnip-shaped, and the like. The balloon portion 18 can preferably inflate to a capacity able to accommodate about 50 to about 200 cc of fluid but any other suitable capacity can alternatively be utilized. The illustrated tube portion 24 has a first lumen 28 that extends from an inflation port 30 to the interior of the balloon portion 18 and a second lumen 32 that extends the length of the balloon catheter 12 from an outer open end 34 to an inner open end 36. The inflation port 30 is configured for connection to the motorized fluid pump 14 with suitable tubing 38 to provide fluid flow communication between the interior of the balloon portion 18 and an inlet/outlet of the motorized fluid pump 14. The tubing 38 can be any suitable type of tubing such as, for example but not limited to, IV tubing, and the like. Any other quantity and/or configuration of lumens can alternatively be utilized. The balloon portion 18 and the tube portion 24 are each preferably formed of biocompatible materials that are elastic in nature such as, for example but not limited to vinyl, latex and the like, but any other suitable material(s) can alternatively be utilized. It is noted that the balloon catheter 12 can alternatively be a Foley catheter or can alternatively have any other suitable configuration.

The illustrated motorized fluid pump 14 is operably connected to the inflation port 30 of the first lumen 28 of the balloon catheter 12 so that it is in fluid flow communication with the interior of the balloon portion 18 of the balloon catheter 12. Connected in this manner, the motorized fluid pump 14 can selectively pump fluid into the balloon portion 18 in order to expand the balloon portion 18 resulting in force being applied by the balloon portion 18 and selectively pump fluid out of the balloon portion 18 in order to contract the balloon portion 18 resulting in less or no force being applied by the balloon portion 18. The illustrated motorized fluid pump 14 can be of any suitable type such as, but not limed to, a liner displacement pump, a rotary displacement pump, and the like. The illustrated motorized fluid pump 14 is electrically powered having a power source 40 in the form of electric mains power, one or more electric battery, and the like. It is noted that the motorized fluid pump 14 can alternatively be powered in any other suitable manner. The illustrated motorized fluid pump 14 is configured to pump saline solution but can alternatively be configured to pump any other suitable type of fluid such as, for example but not limited to, water air, and the like. It is noted that the motorized fluid pump 14 can alternatively have any other suitable configuration.

The illustrated motorized fluid pump 14 is provided with an electronic controller 42 for controlling operation of the motorized fluid pump 14 in a desired manner. The electronic controller 42 is preferably programmed with at least one, and more preferably a plurality, of pre-programmed operational patterns for the motorized fluid pump 14 which can be selected by the operator. For example, but not limited to, the electronic controller 42 can operate the motorized fluid pump 14 to pressurize the balloon portion 18 of the balloon catheter 12 in one or more waveforms. Additionally or alternatively, the electronic controller 42 can be configured to select specific parameters of operation for the motorized fluid pump 14 such as, for example but not limited to, wave shape, amplitude (distance between lowest position and maximum displacement of the wave), frequency (number of waves passing a specific point per second), period (time it takes for one wave cycle to complete), wavelength (distance between adjacent identical parts of a wave, parallel to the direction of propagation), and/or the like. It is noted that the electronic controller 42 can alternatively have any other suitable configuration.

The illustrated system 10 also includes a pressure monitoring device or gauge 44 to indicate the back pressure from the balloon portion 18 of the balloon catheter 12. The pressure monitoring device 44 is operatively connected to the fluid flow path between the motorized fluid pump 14 and the balloon portion 18 of the balloon catheter 12. The pressure monitoring device 44 can provide feedback to the electronic controller 42 and/or a visual indication of current pressure to the operator. It is noted that the pressure monitoring device 44 can be of any suitable type. It is also noted that the pressure monitoring device 44 can alternatively have any other suitable configuration.

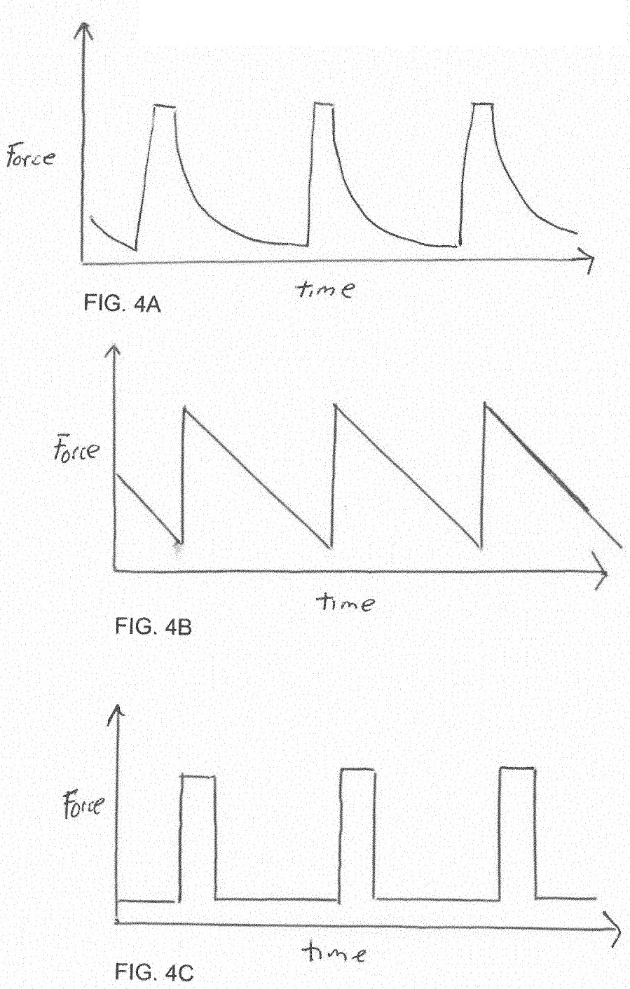

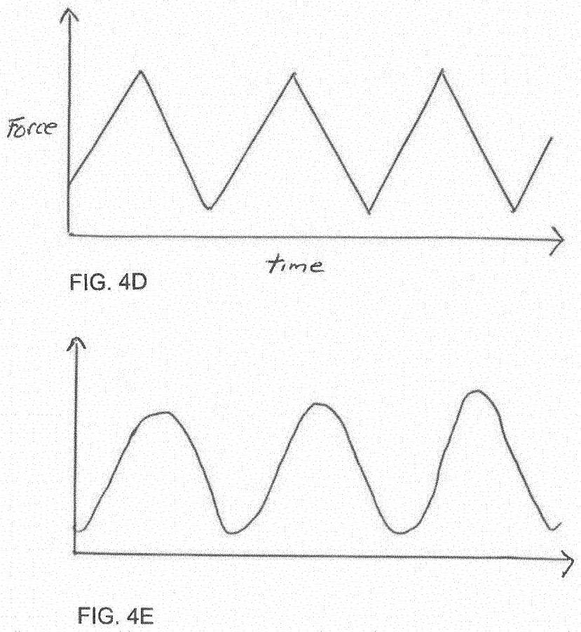

FIG. 4A illustrates an exemplary force versus time on the cervix 20 during natural uterine contractions. The motorized fluid pump 14 is preferably configured to expand and contract the balloon catheter 12 in a repeating manner that pressures the uterine side 22 of the cervix 20 in a manner that simulates these forces during natural uterine contractions due to the baby's head engaging against the uterine side 22 of the cervix 20 during labor. FIGS. 4B to 4B illustrate force versus time of exemplary operation of the motorized fluid pump 14 in the form of waves having saw tooth, square, triangle, and sinusoidal wave shapes respectively. It is noted that the motorized fluid pump 14 can alternatively he configured to operate in any other suitable manner.

The illustrated traction or tension device 16 is configured to provide traction or tension to pull the balloon catheter 12 in an outward direction against the uterine or interior side 22 of the cervix 20. The traction or tension device 16 can be of any suitable type such as, for example but not limited to, adhesive tape or the like securing an exterior portion of the balloon catheter 12 to the woman's leg, a strap or the like securing an exterior portion of the balloon catheter 12 to the woman's leg, a pelvic frame, or the like secured to an exterior portion of the balloon catheter 12, a second balloon portion of the balloon catheter 12 engaging a vaginal or exterior or vaginal side 46 of the cervix 20, and the like. It is noted that the traction or tension device 16 can alternatively have any other suitable configuration or can be eliminated if desired.

In an exemplary method of operation of the system 10, the balloon portion 18 of the balloon catheter 12 is moved through the opening in the cervix 20 so that the balloon portion 18 is located just beyond the cervix 20, but outside the amniotic sac 48. If desired, the outer end of the balloon catheter 12 is secured to the traction or tension device 16. With the port 30 of the first lumen 28 of the balloon catheter 12 connected to the motorized fluid pump 14, the motorized fluid pump 14 is activated to expand and contract the balloon portion 18 of the balloon catheter 12 to repetitively pressure and un-pressure the uterine side 22 of the cervix 20 to simulate forces occurring during natural uterine contractions. With the balloon portion 18 of the balloon catheter 12 inflated, the balloon portion 18 of the balloon catheter 12 puts pressure on the internal side 22 of the cervix 20, helping the cervix 20 dilate and increasing the tissue's response to oxytocin and prostaglandins. With the balloon portion 18 of the balloon catheter 12 contracted, the balloon portion 18 of the balloon catheter 12 puts lower or no pressure on the uterine side 22 of the cervix 20. The balloon catheter 12 usually falls out when the cervix 20 has dilated about 3 cm or more.

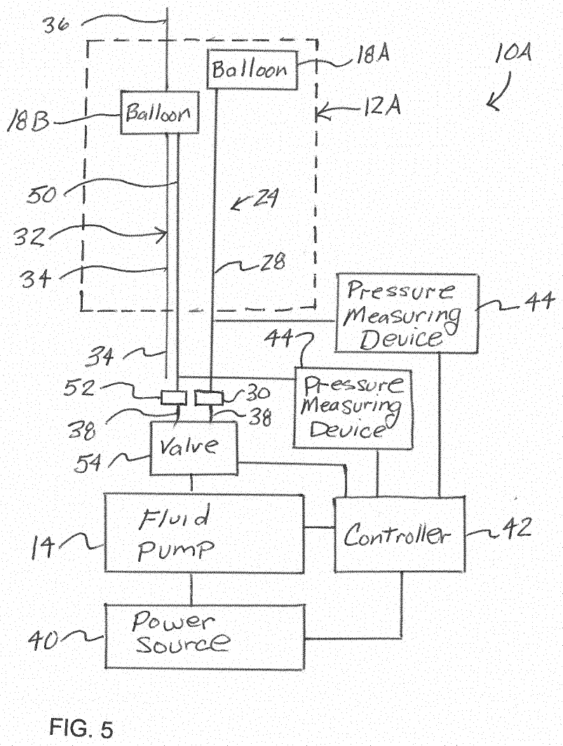

FIGS. 5 to 7 illustrate a cervical ripening system 10A for accelerating cervical ripening according to a second embodiment of the invention. The cervical ripening system 10A according to the second embodiment of the invention is substantially the same as the cervical ripening system 10 according to the first embodiment of the invention described above except that the balloon catheter 12A has two balloon portions 18A, 18B instead of a single balloon portion 18. The first balloon portion 18A is located on the internal or uterine side 22 of the cervix 20 and is repeatedly expanded and contracted by the motorized fluid pump 14 as described above. The second balloon portion 18B is located on the exterior or vaginal side 46 of the cervix 20 and engages the exterior or vaginal side 46 of the cervix 20 to act as the traction or tension device 16. According to a first variation of the second embodiment of the system 10A, the second balloon portion 18B is repeatedly expanded and contracted by the motorized pump 14 as described above instead of the first balloon portion 18A to vary the tension applied onto the first balloon portion 18A in order to vary the force applied to the uterine side 22 of cervix 20 by the first balloon portion 18 A. According to a second variation of the second embodiment of the system 10A, both the first balloon portion 18A and the second balloon portion 18B are repeatedly expanded and contracted by the motorized pump 14 as described above in order to vary the force applied to the uterine side of the cervix 20 by the first balloon portion 18A.

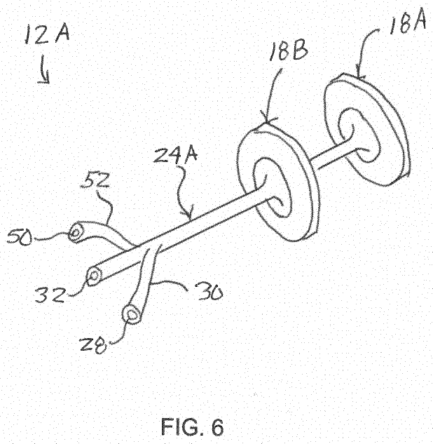

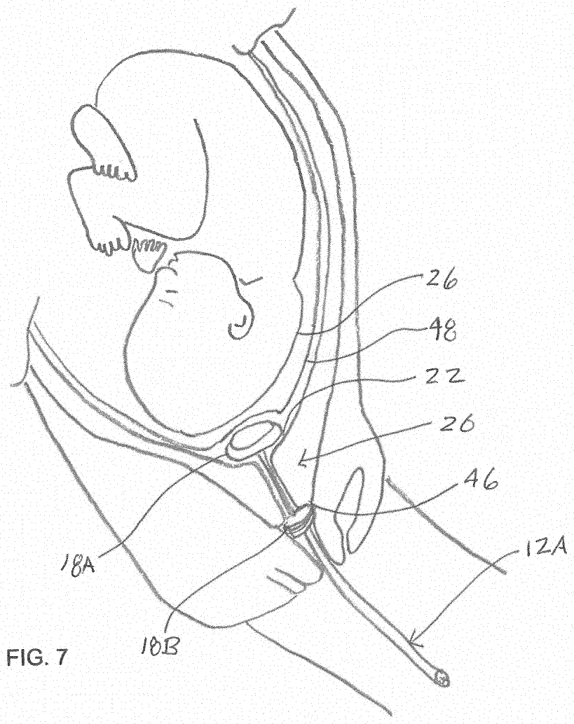

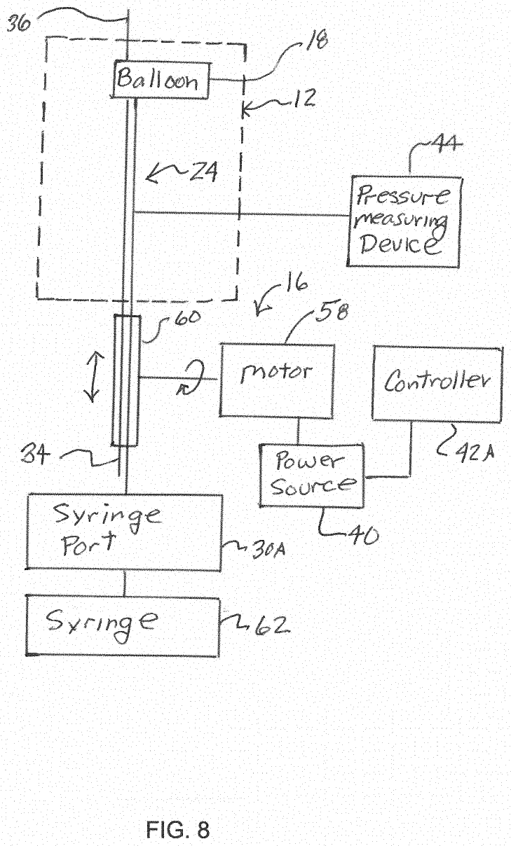

The illustrated two balloon catheter 12A (best seen in FIG. 6) includes the first balloon portion 18A, the second balloon portion 18B, and a tube portion 24A. The illustrated first balloon portion 18A, when inflated, is relatively flat in order to more fully engage the uterine side 22 of the cervix 20 and to minimize interference with the baby's head 26 (best seen in FIG. 7). The illustrated second balloon portion 18B, when inflated, is also relatively flat to more fully engage the exterior or vaginal side 46 of the cervix 20 to tension the first balloon portion (best seen in FIG. 7). However, the first and second balloon portions 18A. 18B can each alternatively have any other suitable shape such as, for example but not limited to, spherical, semi-spherical, cylindrical, oblong or egg-shaped, heart or turnip-shaped, and the like. The first and second balloon portions 18A, 18B can each preferably inflate to a capacity able to accommodate about 50 to about 200 cc of fluid but any other suitable capacity can alternatively be utilized. The illustrated tube portion 24A has a first lumen 28 that extends from an inflation port 30 to the interior of the first balloon portion 18A. a second lumen 32 that extends the length of the balloon catheter 12A from an outer open end 34 to an inner open end 36. and a third lumen 50 that extends from and inflation port 52 to the interior of the second balloon portion 18B. The inflation ports 30, 52 are each configured for connection to the motorized fluid pump 14 with suitable tubing 38 to provide fluid flow communication between the interior of each of the first and second balloon portions 18A, 18B and an inlet/outlet of the motorized fluid pump 14. The tubing 38 can be any suitable type of tubing such as, for example but not limited to, IV tubing, and the like. Any other quantity and/or configuration of lumens can alternatively be utilized. The balloon portions 18A, 18B and the tube portion 24A are each preferably formed of biocompatible materials that are elastic in nature such as, for example but not limited to vinyl, latex and the like, but any other suitable material(s) can alternatively be utilized. It is noted that the balloon catheter 12A can alternatively be a Cook catheter or can alternatively have any other suitable configuration.

The illustrated motorized fluid pump 14 is operably connected to the inflation port 30 of the first lumen 28 of the balloon catheter 12A so that it is in fluid flow communication with the interior of the first balloon portion 18A of the balloon catheter 12A and also to the inflation port 52 of the third lumen 50 of the balloon catheter 12A so that it is also in fluid flow communication with the interior of the second balloon portion 18B of the balloon catheter 12A. A two-way valve 54 is located between the balloon catheter 12A and the motorized pump 14 so that the motorized fluid pump 14 is selectively connected to either the first balloon portion 18A or the second balloon portion 18B. The illustrated valve 54 is an electric valve controlled by the electronic controller 42 but it is noted that the valve 54 can alternatively be of any other suitable type such as, for example but not limited to, a mechanical valve. Connected in this manner, the motorized fluid pump 14 can selectively pump fluid into either the first balloon portion 18A or the second balloon portion 18B in order to expand the selected balloon portion 18A, 18B and can selectively pump fluid out of the either the first balloon portion 18A or the second balloon portion 18B in order to contract the selected balloon portion 18A, 18B as desired. The illustrated motorized fluid pump 14 is as described above with regard to the first embodiment of the system 10. Alternatively, a second motorized fluid pump isoperably connected to the inflation port 52 of the third lumen 50 and the motorized fluid pump is only connected to the inflation port 30 of the first lumen 28. With the two motorized fluid pumps 14 the valve 54 is eliminated and the first and second balloon portions ISA, 18B can be expanded and/or contracted simultaneously.

The illustrated system 10A also includes first and second pressure monitoring devices or gauges 44 to indicate the back pressure from the first balloon portion 18A and the second balloon portion 18B of the balloon catheter 12 A. Alternatively, the single pressure monitoring device or gauge 44 of the first embodiment described above can be connected to each of the first and third lumens 28, 50 via a two valve controlled by the electronic controller 42.

In an exemplary method of operation of the system 10A, the first balloon portion 18A of the balloon catheter 12A is moved through the opening in the cervix 20 so that the first balloon portion 18A is located just beyond the cervix 20, but outside the amniotic sac 48. With the inflation port 30 of the first lumen 28 of the balloon catheter 12A connected to the motorized fluid pump 14, the motorized fluid pump 14 is activated to expand the first balloon portion 18A of the balloon catheter 12 A. Then, with the port 52 of the third lumen 50 of the balloon catheter 12A connected to the motorized fluid pump 14. the motorized fluid pump 14 is activated to expand the second balloon portion 18B of the balloon catheter 12A. Then, with the inflation port 30 of the first lumen 28 of the balloon catheter 12A connected to the motorized fluid pump 14, the motorized fluid pump 14 is activated to expand and contract the first balloon portion 18A of the balloon catheter 12A to repetitively pressure and un-pressure the uterine side 22 of the cervix 20 to simulate forces occurring during natural uterine contractions. With the first and second balloon portions 18A, 18B of the balloon catheter 12A inflated, the first balloon portion 18A of the balloon catheter 12A puts pressure on the internal or uterine side 22 of the cervix 20, helping the cervix 20 dilate and increasing the tissue's response to oxytocin and prostaglandins. With the first balloon portion 18A of the balloon catheter 12A contracted (and/or the second balloon portion 18B of the balloon catheter 12A contracted), the first balloon portion 18A of balloon catheter 12A puts lower or no pressure on the uterine side 22 of the cervix 20. The balloon catheter 12A usually falls out when the cervix 20 has dilated about 3 cm or more.

FIG. 8 illustrates a cervical ripening system 10B for accelerating cervical ripening according to a third embodiment of the invention. The cervical ripening system 10B according to the third embodiment of the invention is substantially the same as the cervical ripening system 10 according to the first embodiment of the invention described above except that instead of using the motorized fluid pump 14 to repetitively pressure and un-pressure the uterine side 22 of the cervix 20 simulating natural uterine contractions, the traction or tension device 16 comprises a motor 58 and rotary-to-reciprocal am version mechanism 60 that are utilized to linearly tension and un-tension the balloon catheter 12, while inflated, in a manner that repetitively pressures and un-pressures the uterine side 22 of the cervix 20 in order to simulate forces occurring during natural uterine contractions. The rotary-to-reciprocal conversion mechanism 60 can be of any suitable type such as, for example but not limited to, a slider-crank mechanism, a rack and pinion mechanism, a screw mechanism, a slotted-link mechanism, a swashplate mechanism, a gear mechanism, and the like. Additionally, the inflation port 30A of the first lumen 28 of the balloon catheter 12 is configured to cooperate with a syringe 62 to selectively inflate the balloon portion 18 of the balloon catheter 12 rather than the motorized fluid pump 14. It is noted, however, that the motorized fluid pump 14 can alternatively be utilized to inflate the balloon portion 18 of the balloon catheter 12 if desired. In a variation of the system 10B of the third embodiment, the motorized fluid pump 14 is utilized to expand and contract the balloon portion 18 of the balloon catheter 12 as described hereinabove in addition to the motor 58 tensioning and un-tensioning the balloon portion 18 of the balloon catheter 12

The illustrated motor 58 can be of any suitable type. The illustrated motor 58 is electrically powered having a power source 40 in the form of electric mains power, one or more electric battery, and the like. It is noted that the motor 58 can alternatively be powered in any other suitable manner. It is noted that the motor 58 can alternatively have any other suitable configuration. The illustrated motor 58 is provided with an electronic controller 42A for controlling operation of the motor 58 in a desired manner. The electronic controller 42A can be configured described hereinabove with regard to the system 10 according to the first embodiment of the invention. It is noted that the electronic controller 42A can alternatively have any other suitable configuration.

In an exemplary method of operation of the system 10B, the balloon portion 18 of the balloon catheter 12 is moved through the opening in the cervix 20 so that the balloon portion 18 is located just beyond the cervix 20, but outside the amniotic sac 48. The inflation port 30A of the first lumen 28 of the balloon catheter 12 is used to expand the balloon portion of the balloon catheter 12 by using a syringe or the like to inject fluid into the balloon portion 18 of the balloon catheter 12. The outer end of the balloon catheter 12 is connected to the motor 58 and the motor 58 is activated to tension and un-tension the balloon portion 18 of the balloon catheter 12 to repetitively pressure and un-pressure the uterine side 22 of the cervix 20 with the balloon portion 18 of the balloon catheter 12 in order to simulate forces that occur during natural uterine contractions. With the balloon portion 18 of the balloon catheter 12 inflated and tensioned, the balloon portion 18 of the balloon catheter 12 puts pressure on the internal side 22 of the cervix 20, helping the cervix 20 dilate and increasing the tissue's response to oxytocin and prostaglandins. With the balloon portion 18 expanded and un-tensioned, the balloon portion 18 of the balloon catheter 12 puts lower or no pressure on the uterine side 22 of the cervix 20. The balloon catheter 12 usually falls out when the cervix 20 has dilated about 3 cm or more.

Any of the features or attributes of the above-described embodiments and variations can be used in combination with any of the other features and attributes of the above-described embodiments and variations as desired.

From the foregoing disclosure it will be apparent that the illustrated systems and methods for accelerating cervical ripening provide greatly reduced times for cervical ripening, and thus the total time of uterine stimulation, in a simple and cost effective manner.

From the foregoing disclosure and detailed description of certain preferred embodiments, it will be apparent that various modifications, additions and other alternative embodiments arc possible without departing from the true scope and spirit of the present invention. The embodiments discussed were chosen and described to provide the best illustration of the principles of the present invention and its practical application to thereby enable one of ordinary skill in the art to utilize the invention in various embodiments and with various modifications as are suited to the particular use contemplated. All such modifications and variations are within the scope of the present invention as determined by the appended claims when interpreted in accordance with the benefit to which they are fairly, legally, and equitably entitled.

What is claimed is:

1. A cervical ripening system comprising, in combination:
a balloon catheter having a balloon portion configured to pass through a cervix when contracted and to engage and pressure a uterine side of the cervix when expanded by a fluid; and
a fluid pump in fluid flow communication with the balloon portion of the catheter and configured to selectively expand and contract the balloon portion of the balloon catheter to repetitively increase and decrease pressure on the uterine side of the cervix.

2. The cervical ripening system according to claim 1, wherein the repetitively increased and decreased pressure on the uterine side of the cervix simulates forces that occur during natural uterine contractions.

3. The cervical ripening system according to claim 1, wherein the repetitively increased and decreased pressure on the uterine side of the cervix is a waveform.

4. The cervical ripening system according to claim 3, wherein the waveform has a shape selected from saw tooth, square, triangle, and sinusoidal.

5. The cervical ripening system according to claim 1, wherein the fluid pump is a motorized fluid pump.

6. The cervical ripening system according to claim 1, further comprising a traction device tensioning the balloon portion against the uterine side of the cervix.

7. A cervical ripening system comprising, in combination:
a balloon catheter having a first balloon portion configured to pass through a cervix when contracted and to engage and pressure a uterine side of the cervix when expanded by a fluid and a second balloon portion configured to pass to a cervix when contracted and to engage a vaginal side of the cervix when expanded by a fluid; and
at least one fluid pump in fluid flow communication with the first balloon portion and the second fluid portion of the catheter and configured to selectively expand and contract at least one of the first and second balloon portions of the balloon catheter to repetitively increase find decrease pressure on the uterine side of the cervix.

8. The cervical ripening system according to claim 7, wherein the repetitively increased and decreased pressure on the uterine side of the cervix simulates forces that occur during natural uterine contractions.

9. The cervical ripening system according to claim 7, wherein the repetitively increased and decreased pressure on the uterine side of the cervix is a waveform.

10. The cervical ripening system according to claim 9, wherein the waveform has a shape selected from saw tooth, square, triangle, and sinusoidal.

11. The cervical ripening system according to claim 7, wherein the fluid pump is a motorized fluid pump.

12. The cervical ripening system according to claim 7, wherein the second balloon portion engages the vaginal side of tire cervix and acts as a traction device to tension the first balloon portion against the uterine side of the cervix.

13. The cervical ripening device according to claim 7, wherein the at least one pump selectively expands and contracts only the first balloon portion.

14. The cervical ripening device according to claim 7, wherein the at least one pump selectively expands and contracts only the second balloon portion.

15. A method for cervical ripening comprising the steps of, in combination:
passing a balloon portion of a balloon catheter through a cervix and into a uterus when the balloon portion is contracted; and
expanding and contracting the balloon portion of the balloon catheter within the uterus with a fluid using a fluid pump in fluid flow communication with the balloon portion of the balloon catheter to repetitively increase and decrease pressure on the uterine side of the cervix.

16. The method for cervical ripening according to claim 15, wherein the repetitively increased and decreased pressure on the uterine side of the cervix simulates forces that occur during natural uterine contractions.

17. The method for cervical ripening according to claim 15, wherein the repetitively increased and decreased pressure on the uterine side of the cervix is a waveform.

18. The method for cervical ripening according to claim 17, wherein the waveform has a shape selected from saw tooth, square, triangle, and sinusoidal.

19. The method for cervical ripening according to claim 15, wherein the fluid pump is a motorized fluid pump.

20. The method for cervical ripening according to claim 15, further comprising a traction device tensioning the balloon portion against the uterine side of the cervix.
